(19) United States
(12) Patent Application Publication
(10) Pub. No.: US 2025/0170384 A1
(43) Pub. Date: May 29, 2025

(54) HUB ATTACHMENT COLLAR FOR SURGICAL CATHETER SYSTEMS AND METHODS FOR USE THEREOF

(71) Applicant: CANON U.S.A., INC., MELVILLE, NY (US)

(72) Inventors: Takahisa Kato, Brookline, MA (US); Zachary Hamilton Haubert, Somerville, MA (US); Charles George Hwang, Wellesley, MA (US)

(21) Appl. No.: 18/947,878

(22) Filed: Nov. 14, 2024

Related U.S. Application Data
(60) Provisional application No. 63/603,365, filed on Nov. 28, 2023.

(51) Int. Cl. A61M 39/10

(52) U.S. Cl. CPC A61M 39/1055; A61M 39/1011; A61M 2039/1016; A61M 2039/1044; A61M 2039/1066; A61M 2205/3327; A61M 2205/581; A61M 2205/582; A61M 2205/583

(57) ABSTRACT

Disclosed are an apparatus and method for medical applications and, more particularly, a hub attachment collar applicable for safe disconnect of guide tools and devices used in medical procedures, including in robotic medical procedures, with such guide tools and devices including catheters, cameras, and endoscopes.

### CROSS-REFERENCE TO RELATED APPLICATION(S)

The present application claims priority to U.S. Provisional Patent Application No. 63/603,365, which was filed with the U.S. Patent and Trademark Office on Nov. 28, 2023, the entire disclosure of which is incorporated herein by reference.

### BACKGROUND

### 1. Field

The present disclosure relates generally to devices and methods for medical applications and, more particularly, to a hub attachment collar applicable for safe disconnect of guide tools and devices used in medical procedures, including in robotic medical procedures, with such guide tools and devices including catheters, cameras, and endoscopes.

### 2. Related Art

Flexible medical instruments such as endoscopic surgical devices and catheters are broadly used in surgical and probative settings. Such flexible medical instruments continue to gain acceptance in the medical field. The medical devices generally include a flexible tube commonly referred to as a sleeve or sheath, with one or more tool channels extending along or inside the sheath to allow access to end effectors located at a distal end of the sheath.

Conventional systems include U.S. Pub. 2021/0259794 to Kato et al., entitled Medical apparatus having dual manipulation means and methods for use thereof; U.S. Pat. 11,559,190 to Okumura et al., entitled Steerable medical device and method; and U.S. Pub. 2021/0369085 to Kato et al., entitled Medical apparatus with reflow trapped anchors and method of use thereof, the content of each of which is incorporated herein by reference.

EP 2589407B1 discusses a catheter system with an attachable catheter hub via rotation around the hub for attachment. U.S. Pat. 11,553,917 to Sgroi discusses a loading unit locking collar with rotational actuated release. JP6857283B2 discusses a handheld surface cleaning unit in which components are mated together via tab flexes into a slot to lock components, with push tab release. JP2023103865A and U.S. Pub. 2021/0121051 discuss a medical device and bendable unit. JP 2022-115015 discusses a medical apparatus having a main body that includes a plurality of drive sources, and an operation unit movable between a fixed position and a detached position when a bendable unit is attached to a base station.

### SUMMARY

However, conventional systems fail to provide a system with an attachment collar that connects a catheter to a medical apparatus actuator, including an inaction zone, a wire disengagement zone, and a hub release zone to provide safety and avoid accidental turning. To overcome various shortcomings of conventional systems, the present disclosure provides a hub attachment collar, also referred to as collar herein, configured to connect a catheter to a medical apparatus actuator, for example a robotic catheter system for peripheral lung biopsy.

An aspect of the present disclosure provides an attachment collar that detachably connects a medical apparatus actuator to a catheter hub, with the attachment collar including an inaction zone, a wire disengagement zone, and a hub release zone.

Another aspect of the present disclosure provides a method for securing a catheter to an actuator of a medical apparatus, with the catheter including a catheter hub, and with the actuator including a handle and an attachment collar operable through an inaction zone, a wire disengagement zone, and a hub release zone, and with the method including attaching, with the attachment collar in the hub release zone, the handle to the catheter hub; rotating the attachment collar through the hub release zone into the wire disengagement zone; and rotating the attachment collar through the wire disengagement zone into the inaction zone, with the rotating through the wire disengagement zone securing attachment pins extending from the catheter hub to respective clamping mechanisms of the actuator.

A further aspect of the present disclosure provides a method for releasing a catheter from an actuator of a medical apparatus, the catheter including a catheter hub; the actuator including a handle and an attachment collar operable through an inaction zone, a wire disengagement zone, and a hub release zone; and with the method including exerting a rotational force on the attachment collar to rotate the attachment collar through the inaction zone to a detent provided at a transition between the inaction zone and the wire disengagement zone; receiving feedback from the detent; in response to the feedback, exerting one of a pressing force and an increased rotational force to transition from the inaction zone to the wire disengagement zone; rotating the attachment collar through the wire disengagement zone to a hard stop provided at a transition between the wire disengagement zone and the hub release zone; rotating past the hard stop by activating a release mechanism; rotating the attachment collar through the hub release zone, with the rotating of the attachment collar through the hub release zone releasing attachment pins extending from the catheter hub from respective clamping mechanisms of the actuator; and detaching the handle from the catheter hub.

### BRIEF DESCRIPTION OF THE DRAWINGS

The accompanying drawings, which are incorporated herein and form part of the specification, illustrate various embodiments, objects, features, and advantages of the present disclosure.

FIG. 1 illustrates a medical environment for use of a robotic catheter system, according to an embodiment.

FIG. 2 illustrates components of the robotic catheter system, according to an embodiment.

FIG. 3 illustrates a steerable catheter, according to an embodiment.

FIG. 4 is perspective view of an actuation handle of robotic catheter system of a medical apparatus, combined with a catheter hub, with a hub attachment collar therebetween, according to an embodiment.

FIG. 5 illustrates zones of movement of the hub attachment collar, according to an embodiment.

FIG. 6A illustrates an external locking collar and an internal locking collar provided within the catheter hub, according to an embodiment.

FIG. 6B is a cut away view of FIG. 6A illustrating clamping mechanisms within the attachment collar, according to an embodiment.

FIGS. 7A to 7D illustrate a process for attaching the catheter hub to the actuation handle, according to an embodiment.

FIG. 8 illustrates a shelf mechanism for aligning the catheter hub with the attachment collar, according to an embodiment.

FIG. 9 illustrates a rail mechanism for aligning the catheter hub with the attachment collar, according to an embodiment.

FIGS. 10A to 10D illustrate a process for detaching the catheter hub from the actuation handle, according to an embodiment.

FIGS. 11A and 11B illustrate a sleeve guard, according to an embodiment.

FIG. 12 illustrates operation of a release mechanism, according to an embodiment.

FIG. 13 illustrates a method for securing a catheter to an actuator of a medical apparatus, according to an embodiment.

FIG. 14 illustrates a method for releasing a catheter from an actuator of a medical apparatus, according to an embodiment.

### DETAILED DESCRIPTION OF EXEMPLARY EMBODIMENTS

Aspects of the present disclosure can be understood by reading the following detailed description in light of the accompanying figures. It is noted that, in accordance with standard practice, the various features of the drawings are not drawn to scale and do not represent actual components. Several details such as dimensions of the various features may be arbitrarily increased or reduced for ease of illustration. In addition, reference numerals, labels and/or letters are repeated in the various examples to depict similar components and/or functionality. This repetition is for the purpose of simplicity and clarity and does not in itself limit the various embodiments and/or configurations the same components discussed.

Before the various embodiments are described in further detail, it shall be understood that the present disclosure is not limited to any particular embodiment. It is also to be understood that the terminology used herein is for the purpose of describing exemplary embodiments only, and is not intended to be limiting. Embodiments of the present disclosure may have many applications within the field of medical treatment or minimally invasive surgery (MIS).

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of introduced claim recitation(s) is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As used herein, the term “real-time” is meant to describe processes or events communicated, shown, presented, etc. substantially at the same time as those processes or events actually occur. Real time refers to a level of computer responsiveness that a user senses as sufficiently immediate or that enables the computer to keep up with some external process. For example, in computer technology, the term real-time refers to the actual time during which something takes place and the computer may at least partly process the data in real time (as it comes in). As another example, in signal processing, “real-time” processing relates to a system in which input data is processed within milliseconds so that it is available virtually immediately as feedback, e.g., in a missile guidance, an airline booking system, or the stock market real-time quotes (RTQs).

The present disclosure generally relates to medical devices, and it exemplifies embodiments of an endoscope or catheter, and more particular to a steerable catheter controlled by a medical continuum robot (MCR). The embodiments of the endoscope or catheter and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

As known in the field of medical devices, the terms “proximal” and “distal” are used with reference to the manipulation of an end of an instrument extending from the user to a surgical or diagnostic site. In this regard, the term “proximal” refers to the portion of the instrument that is closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute. In that regard, all directional references (e.g., upper, lower, upward, downward, left, tight, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure.

As used herein the term “catheter” generally refers to a flexible and thin tubular instrument made of medical grade material designed to be inserted through a narrow opening into an anatomical bodily lumen (e.g., an airway or a vessel) to perform a broad range of medical functions. The more specific term “steerable catheter” refers to a medical instrument comprising an elongated flexible shaft having at least one tool channel spanning through a plurality of bendable segments that are actuated by an actuator that applies an actuation force via drive wires arranged along a wall of the shaft.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, the terms “optical fiber”, “fiber optic”, or simply “fiber” refers to an elongated, flexible, light conducting waveguide capable of conducting light from one end to another end due to the effect known as total internal reflection. The terms “light guiding component” or “waveguide” may also refer to, or may have the functionality of, an optical fiber. The term “fiber” may refer to one or more light conducting fibers.

### Robotic Catheter System

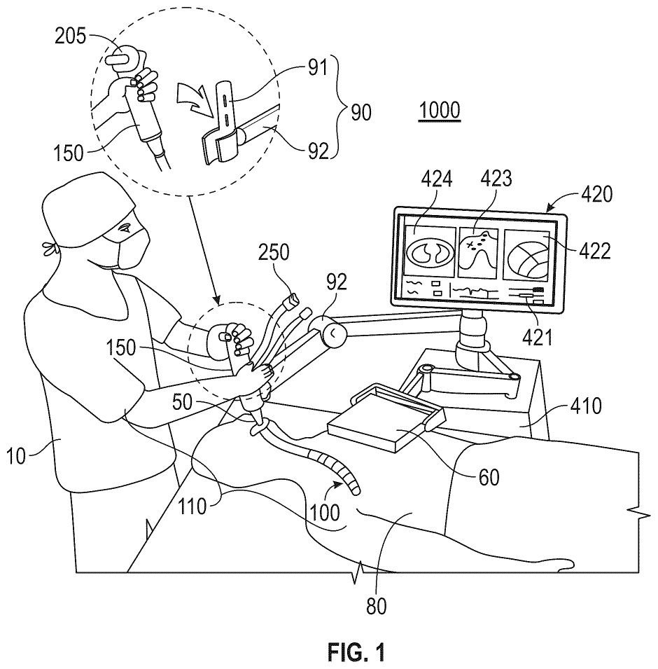

An exemplary configuration of a robotic catheter system 1000 is described with reference to FIGS. 1 to 3.

FIG. 1 illustrates a medical environment for use of a robotic catheter system. As shown in FIG. 1, a robotic catheter system 1000 may be used in an operating room as, e.g., the medical environment. The system 1000 may include a robotic catheter 110 operable by a user 10 (e.g., a physician) to perform an intraluminal procedure on a patient 80. The system 1000 may include a computer 400 operatively connected to the robotic catheter 110 via a robotic platform 90. The robotic platform 90 includes one or more than one robotic arm 92 and a translation linear stage 91. The computer 400 (e.g., a system console) includes at least a central processing unit (CPU) 410 comprised of one or more than one processor, and a display screen 420 (display device) such as a liquid crystal display (LCD), OLED or QLED display.

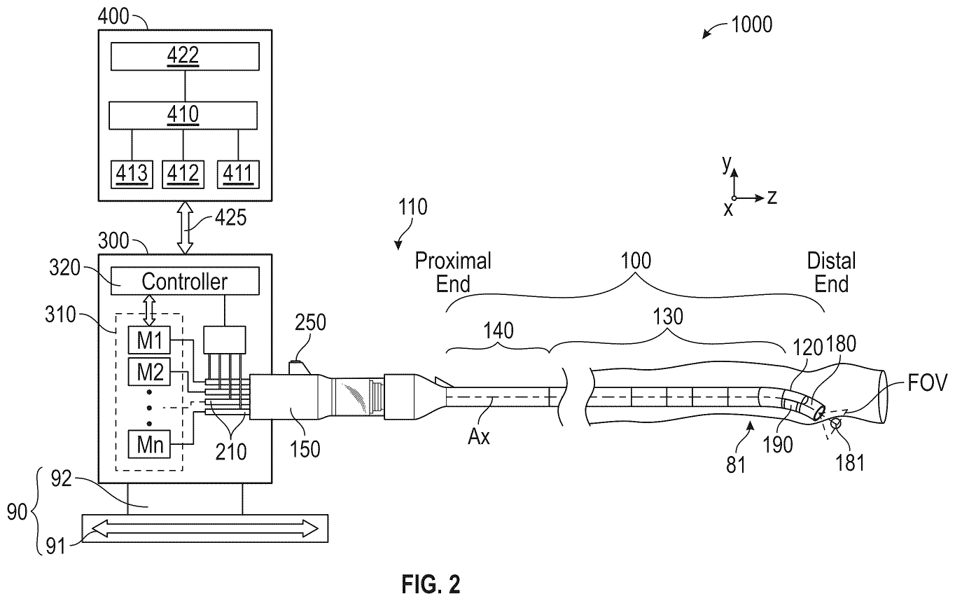

FIG. 2 illustrates components of the robotic catheter system. As shown in FIG. 2, the CPU 410 is operatively connected to a storage memory 411 (ROM and RAM memory), a system interface 412 (e.g., FPGA card), a user interface 413 (e.g. mouse and keyboard), and to the display screen 420.

The robotic catheter 110 includes an actuation handle 150 and a steerable catheter 100. The steerable catheter 100 is removably attached to the actuation handle 150 via a connector assembly 50 (connector hub). The steerable catheter 100 may also be referred to as a continuum robot catheter or a snake robot catheter configured to form continuous curves based on actuation principles known in the art. A well-known approach to form continuous curves with a continuum robot catheter is the follow-the-leader (FTL) technique. The actuation handle 150 connects to an actuator system 300 which receives electronic commands from the computer 400 to mechanically actuate the steerable catheter 100. The actuation handle 150 is configured to be detachably mounted on the robotic platform 90 for robotically guiding the steerable catheter 100 through a bodily lumen 81 towards a target 181 within the subject or patient 80. When the actuation handle 150 is not mounted on the robotic platform 90, the actuation handle 150 can be operated manually by the user 10 one or more knobs to control the steerable catheter 100. For treating or examining a patient 80, the robotic catheter 110 may include one or more access ports 250 arranged in or around the actuation handle 150. Access ports 250 are used to introduce end effector tools, or to pass fluids to/from the patient 80. A tracking system (including, e.g., an electromagnetic (EM) field generator 60 and one or more EM sensors 190 arranged on the steerable catheter 100) is used for tracking the position, shape, pose, and/or orientation of the steerable catheter 100 while being inserted through the bodily lumen 81 towards the target 181. The target 181 is a region of interest (e.g., center of a tumor or a lesion) located in or around the lumen 81 of the patient 80. Alternatively or in addition to EM components, the tracking system may include magnetic and/or radiopaque markers.

During an intraluminal procedure, the system's processor or CPU 410 is configured to perform operations based on the user's input by executing (processing) computer-executable code pre-stored in the system's memory 411. The display screen 420 may include a graphical user interface (GUI) configured to display a graphical representation 421 of catheter navigation parameters and patient information, an endoscope image 422 (live view image), an intra-operative guiding image 423, and a pre-operative image 424 (e.g., a 3D or 2D slice image) of a region of interest of the patient 80. Intra-operative guiding image 423 may include conventional fluoroscopy images, or acoustic or ultrasound images. Pre-operative image 424 may include 2D or 3D computed tomography (CT) or magnetic resonance imaging (MRI) images.

As shown in FIG. 2, the steerable catheter 100 is comprised of a proximal section 140, a distal section 130, and a rigid catheter tip 120 arranged in this order from the proximal end to the distal end along a catheter axis (Ax). The distal section 130 is a steerable section comprised of a plurality of bendable segments. The proximal section 140 includes a flexible non-steerable tubular shaft which serves to connect the steerable section 130 to the actuation handle 150. At least the catheter tip 120 includes the tracking sensor 190 (e.g., one or more EM sensors), which is tracked by the system 1000 based on an EM field generator 60.

The steerable catheter 100 is controlled by an actuation system comprised of the actuation handle 150, the actuator system 300, the robotic platform 90 and/or a handheld controller 205 (e.g., a gamepad controller or joystick), which are in electronic communication with the computer 400 via a cable or network connection 425. The actuator system 300 includes a micro-controller 320 and an actuator 310 which are operatively connected to the computer 400 via the network connection 425. The micro-controller 320 may include a proportional-integral-derivative (PID) controller or other similar digital signal processor (DSP) circuit. The actuator 310 includes a plurality of actuating servo motors (or piezoelectric actuators) M1 through Mn, where “n” can be equal to a number of drive wires 210 necessary for steering the steerable catheter 100.

The robotic control system 300 also includes one or more sensors, including strain, position, and/or locked state sensors. The strain sensor can be implemented by, for example, a strain gauge or a piezo resistor. A strain sensor serves to detect and/or measure compressive or tensile forces exerted on each drive wire 210. In this case, the strain sensor outputs a signal corresponding to the amount of compressive or tensile force (an amount of strain) being applied to each drive wire 210 during actuation of the steerable catheter 100. The sensors may output a signal corresponding to an amount of movement (distance of displacement) for each actuated drive wire 210. A sensor may measure the amount of displacement of the drive wire may also be implemented by a Hall-effect sensor. A sensor may be part of a tracking system implemented by an electromagnetic (EM) sensor configured to measure and/or detected the position and orientation (pose) of the catheter tip 120. Signals from the sensors (strain sensor, displacement sensor, and/or pose or position sensor) for one or more drive wires 210 are sent to the controller 320 and/or computer 400 to provide real-time feedback and create closed-loop control for each motor or actuator. In this manner, each drive wire 210 can be actively controlled to implement appropriate shaft guidance for safely navigating the steerable catheter 100 through the lumen 81.

The computer 400 includes suitable software, firmware, and peripheral hardware operated by one or more processor of CPU 410. The computer 400, the actuator system 300, and the actuation handle 150 are operably connected to each other by the network connection 425 (e.g., a cable bundle or wireless link). In addition, the computer 400, the actuator system 300 and the actuation handle 150 are operatively connected to each other by the robot platform 90. In some embodiments, the actuator system 300 may include or be connected to a handheld controller, such as a gamepad controller or a portable computing device like a smart phone or a tablet. Among other functions, the computer 400 and actuator system 300 can provide a surgeon or other operator with a GUI and navigation information through the display screen 420 to operate the steerable catheter 100.

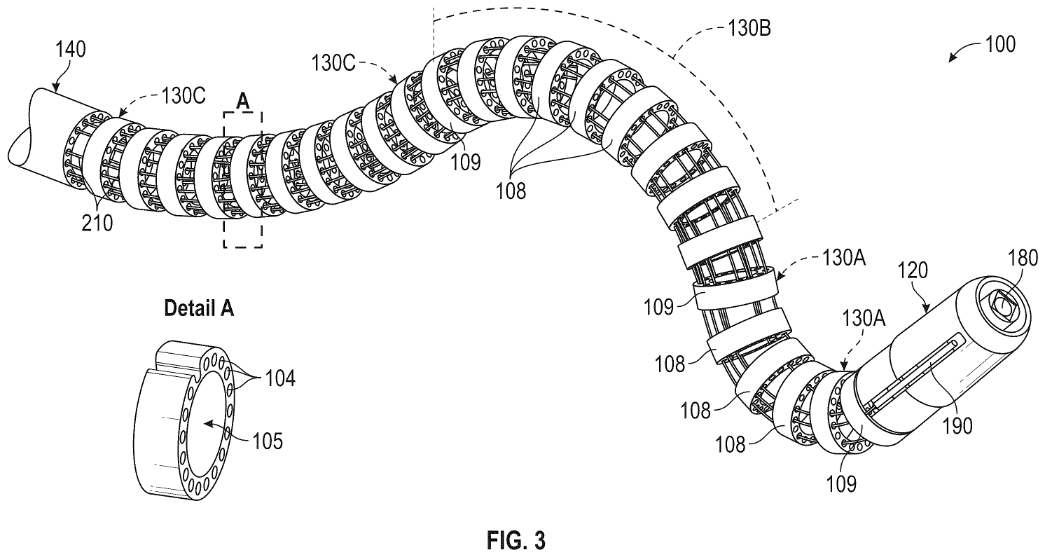

FIG. 3 illustrates a steerable catheter 100. The proximal section 140 is configured to be attached to the actuation handle 150 via the connector assembly 50. The steerable distal section 130 includes a plurality of bendable segments configured to be actuated by drive wires 210 arranged along the wall of the catheter. The bendable segments of the steerable catheter 100 may include a distal bendable segment 130A, a middle bendable segment 130B, and a proximal bendable segment 130C. Each bendable segment is formed by a plurality of ring-shaped components (rings). The ring-shaped components are defined as wire-guiding members 108 or anchor members 109 depending on their function within the catheter. Anchor members 109 are ring-shaped components onto which the distal end of one or more drive wires 210 are attached. Wire-guiding members 108 are ring-shaped components through which some drive wires 210 slide through (without being attached thereto).

Detail A in FIG. 3 illustrates an exemplary embodiment of a ring-shaped component (a wire-guiding member 108 or an anchor member 109). Each ring-shaped component may include a central opening which forms the tool channel 105, and plural conduits 104 (sub-channels, or thru-holes) formed lengthwise and arranged equidistant from the central opening along the annular wall of each ring-shaped component. The non-steerable proximal section 140 is a tubular shaft made of extruded polymer material. The tubular shaft of the proximal section 140 may also have a central opening or tool channel 105 and plural conduits 104 along the wall of the shaft surrounding the tool channel. In this manner, at least one tool channel 105 formed inside the steerable catheter 100 provides passage for an imaging device 180 and/or end effector tools from the access ports 250 to the distal end of the catheter.

An imaging device 180 that can be inserted through the tool channel includes an endoscope camera (videoscope) along with illumination optics (e.g., optical fibers or LEDs). The illumination optics provides illumination light to irradiate a lesion target 181 which is a region of interest within the patient. End effector tools refer endoscopic surgical tools including clamps, graspers, scissors, staplers, ablation or biopsy needles, and other similar tools, which serve to manipulate body parts (organs or tumorous tissue) during examination or surgery.

Next, an example of robotic navigation of the steerable catheter 100 is explained. In general, either during insertion or retraction of the steerable catheter 100 through a bodily lumen 81, the center line of the lumen (e.g., the center line of a lung's airway) is considered the desired trajectory to be followed during active control of the bendable segments of the steerable section 130 (refer to FIG. 3). To that end, various kinematic techniques are used to robotically operate the steerable catheter 100 with the goal of controlling the bendable segments to guide the catheter tip 120 along the desired trajectory to reach a target. In one such example, during robotic navigation, the steerable catheter 100 is advanced through a lumen 81 while sensors measure the insertion depth of the catheter shaft, monitor the force applied to the catheter, and measure angulations of the catheter tip to obtain trajectory information. The trajectory information is stored in a memory of the system and continuously updated. After a short advance in insertion depth, the shape of the steerable catheter is updated by steering (rotating, twisting, or bending) one or more of the bendable segments of the catheter in such a way that the new shape closely matches the desired trajectory. This process is repeated until the target is reached. The same process (in reverse) is applied when the steerable catheter is withdrawn from the patient's lumen. The segments of the distal steerable section 130 can be controlled individually to direct the catheter tip 120 with a combined actuation of all bendable segments, or the catheter tip can be operated in an FTL approach by controlling the most distal segment and the remaining segments following the path traced out by the most distal segment. To withdraw the catheter, a reverse FTL (rFTL) process can be implemented.

In a robotic catheter system as described herein, catheter collision with the patient's anatomy may occur when the catheter trajectory is not maintained within the constraints of the lumen. In general, when navigating along a straight section of a lumen, it is desirable to maintain the catheter along the center line of the lumen. If making a turn, the trajectory should be offset away from the center line to navigate “around the corner” of a tight curve, in particular when the catheter has a rigid catheter tip. Therefore, when navigating through tortuous anatomies, the approach path that was followed by the catheter tip can deviate from the intended trajectory for various reasons (e.g., patient movement, user intervention, or the like). User-guided deviation from an ideal path (insertion trajectory) can propagate from the distal most section to the subsequent bendable segments based on the control algorithm used. In addition, the position of the robotically controlled bendable segments can deviate from the user-guided path for numerous reasons, including different section designs, different tolerances for different bendable segments, different positions of the base of that section, etc.

Challenges may arise during navigation due to a number of reasons, as described in the documents disclosed by the related art, including delays that may result from retracting the catheter 100 and re-start of a surgical procedure, which adds unwanted time to the surgical procedure.

### Hub Attachment Collar

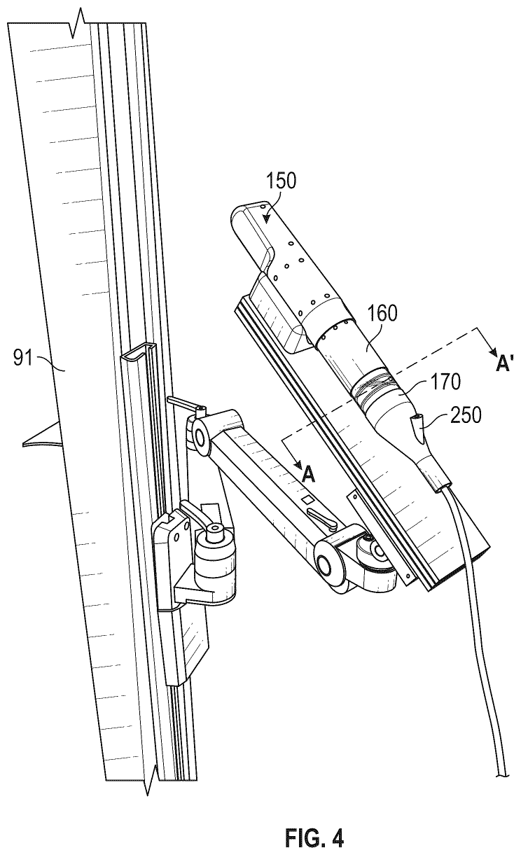

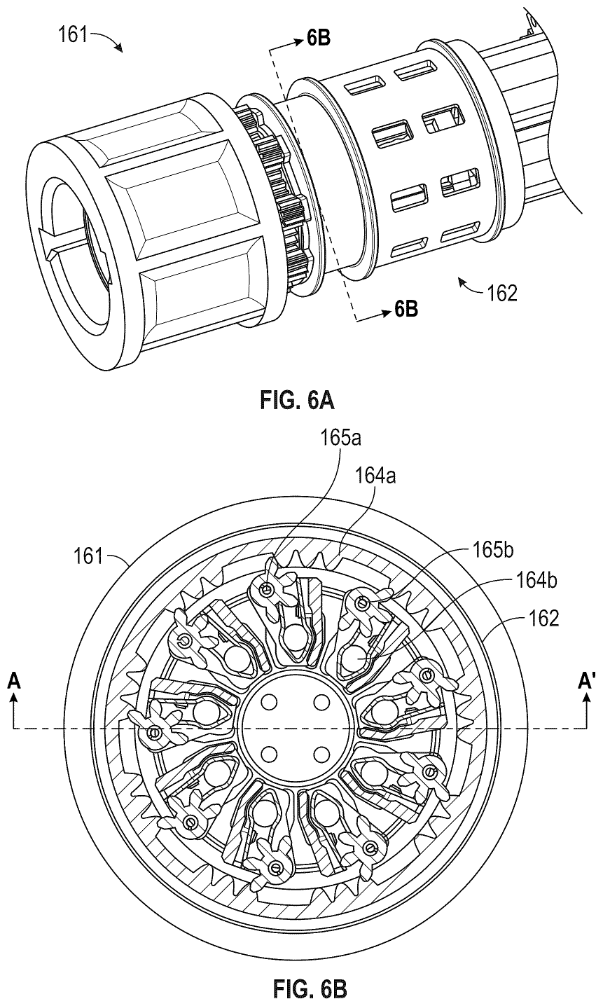

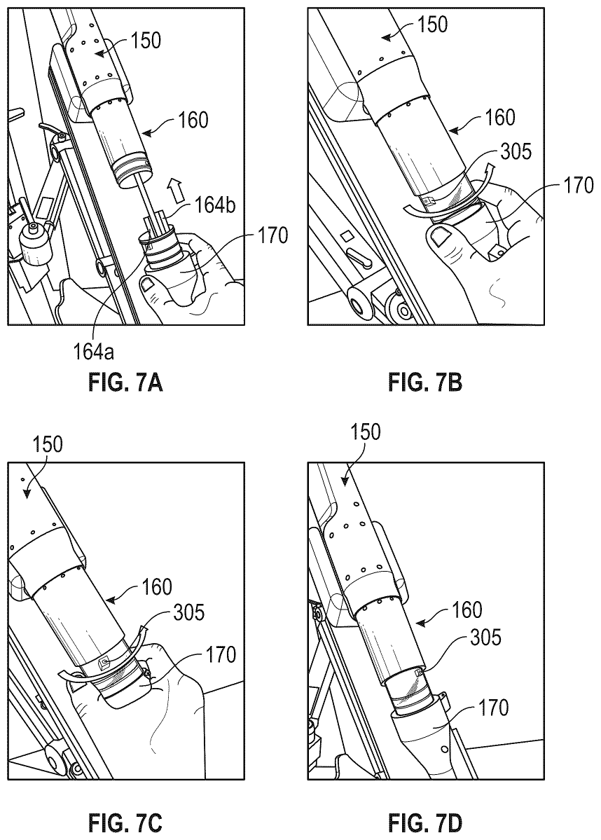

FIG. 4 is perspective view of the actuation handle 150 of the robotic catheter system 1000 of the medical apparatus, combined with a catheter hub 170, with a hub attachment collar 160 (also referred to as attachment collar or collar herein) therebetween, according to an embodiment. As illustrated in FIG. 4, the collar 160 may be provided between the actuation handle 150 and the catheter hub 170. That is, the attachment collar 160 may be rotatably connected to the actuation handle 150, to control attachment/release of attachment pins 164a, 164b (FIG. 7A) to/from respective clamping mechanisms 165a, 165b (FIG. 6B), which are connected to respective tractors of the actuator system 300.

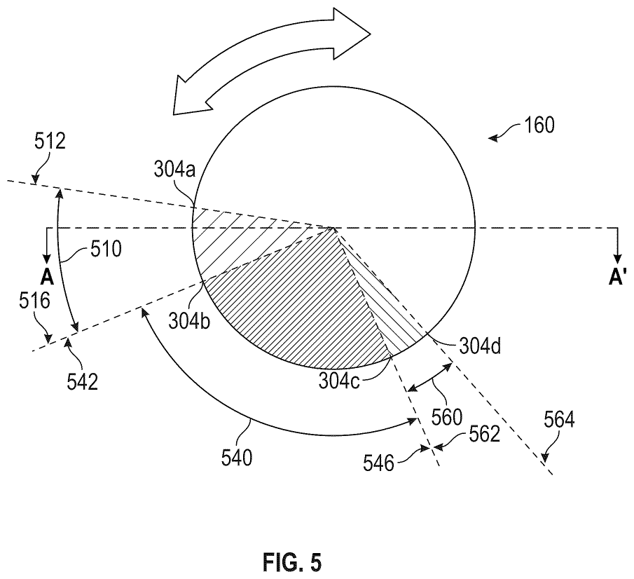

FIG. 5 illustrates zones of movement of the collar 160, according to an embodiment. FIG. 5 is a cut away view along line A-A′ of FIG. 4.

As illustrated in FIG. 5, the zones of movement include an inaction zone 510, a wire disengagement zone 540, and a hub release zone 560. The inaction zone 510 may occupy ranges of one or more of approximately ten degrees of rotation, approximately five to fifteen degrees of rotation, and approximately ten to forty-five degrees of rotation, with the relative degrees of rotation being variable depending on factors that include a number of total drive wires and respective clamping mechanisms 165a, 165b.

The collar 160 may be rotated relative to at least one of the actuation handle 150 and the catheter hub 170. In an embodiment, rotational movement of the actuation handle 150 controls attachment/release of the attachment pins 164a, 164b to/from the respective clamping mechanisms 165a, 165b.

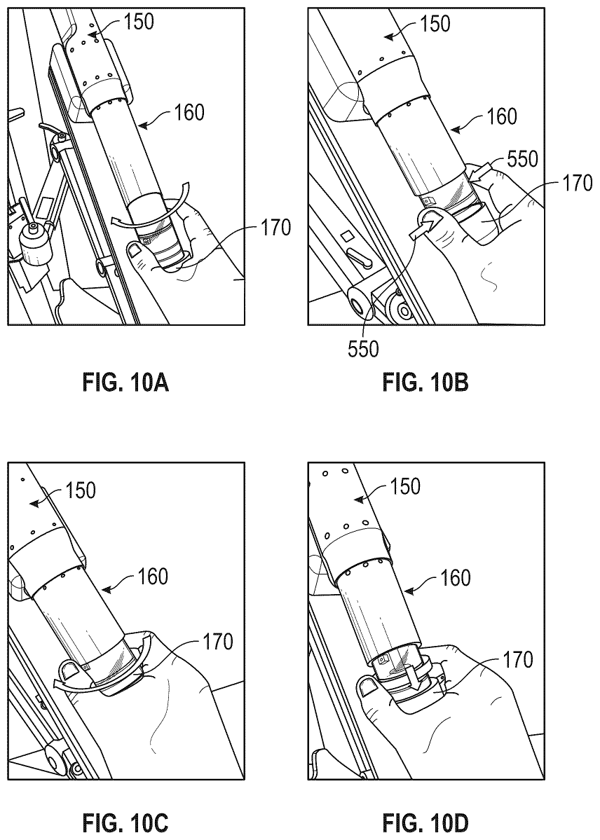

A user may rotate the collar 160 in a release direction from the inaction zone 510, through the wire disengagement zone 540, and into the hub release zone 560 (FIGS. 10A-10D). The user may rotate the collar 160 in a securing direction from the hub release zone 560, through the wire disengagement zone 540, and into the inaction zone 510 (FIGS. 7A-7D). The hub release zone 560 may occupy approximately ten degrees of rotation, which may vary depending on factors that include a number of total drive wires and respective clamping mechanisms 165a, 165b.

Rotation of the collar 160 through the wire disengagement zone 540 facilitates attachment/detachment of a medical apparatus actuator 310 to/from the catheter hub 170. The wire disengagement zone 540 may occupy ranges of one or more of approximately 90 degrees of rotation, approximately eighty to one hundred degrees of rotation, and approximately sixty to one hundred and twenty degrees of rotation, which may vary depending on factors that include a number of total drive wires and respective clamping mechanisms 165a, 165b.

The inaction zone 510 includes a first end 512 and a second end 516 that is opposite the first end 512. The first end 512 of the inaction zone 510 may include a first hard stop to preclude rotation past the first end 512 of the inaction zone 510. Rotation of the attachment collar 160 between the first end 512 and the second end 516 of the inaction zone 510 does not result in a change of state or action being triggered by the attachment collar 160. Prior to rotation, the attachment collar 160 is maintained in a stationary state by a detent that is located at the first end 512 of the inaction zone 510.

When in the inaction zone 510, attachment pins 164a, 164b (FIG. 7A) of the catheter hub 170 are secured from the attachment collar 160, for example by respective clamping mechanisms 165a, 165b (FIG. 6B). Rotation of the attachment collar 160 in the release direction past the second end 516 of the inaction zone 510 may be arrested by a second detent or similar structure that provides to the user of the attachment collar 160 at least one of tactile feedback and/or audio feedback. The detent may be a virtual detent or may be a mechanical detent, e.g., a notched wheel, a spring actuated lever, or the like. The detent may provide at least one of vibrotactile feedback and/or require increased rotational force to continue rotation of the attachment collar 160 past the second end 516 of the inaction zone 510 into the wire disengagement zone 540. The feedback may also include one or more of an audible sound, a visual indicator.

The wire disengagement zone 540 may include a first end 542 and a second end 546, with the first end 542 of the wire disengagement zone 540 being substantially adjacent to the second end 516 of the inaction zone 510 with the second detent therebetween.

The second end 546 of the wire disengagement zone 540 may include a second hard stop that impedes rotation in the release direction of the attachment collar 160 past the second end 546 of the wire disengagement zone 540 until the user activates a release mechanism 550. The second hard stop that is provided at the second end 546 of the wire disengagement zone 540 may prevent accidental, continued rotation into the hub release zone 560. A first end 562 of the hub release zone 560 may be substantially adjacent to the second end 546 of the wire disengagement zone 540 with the second hard stop therebetween.

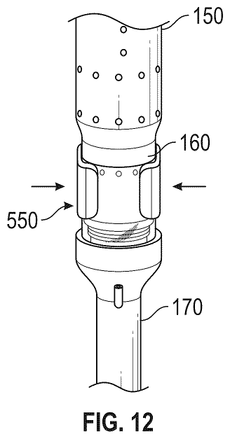

The release mechanism 550 may be a push button or engagement latch and an example of the release mechanism 550 is provided in FIG. 12. The release mechanism 550 avoids inadvertent release of the catheter hub 170, which may require restarting the surgical procedure from the beginning, thereby adding unwanted time to the surgical procedure.

At the end of such rotation, the attachment collar 160 is maintained in the stationary state by a detent that is located at a second end 564 of the hub release zone 560.

FIG. 6A illustrates an external locking collar and an internal locking collar provided within the catheter hub, according to an embodiment. FIG. 6B is a cut away view of FIG. 6A illustrating clamping mechanisms within the attachment collar, according to an embodiment.

As illustrated in FIGS. 6A and 6B, the attachment collar 160 includes an external user cylinder 161 and an internal cylinder 162 configured to secure attachment pins 164a, 164b (FIG. 7A) that extend from a distal end of the catheter hub 170 by respective clamping mechanisms 165a, 165b.

FIGS. 7A to 7D illustrate a process for attaching the catheter hub 170 to the actuation handle 150, according to an embodiment.

In FIG. 7A, the catheter hub 170 is separated from the actuation handle 150, with the attachment collar 160 positioned in the hub release zone 560. Then, the catheter hub 170 is moved axially toward the actuation handle 150, and the plurality of drive wires 210 also move toward the actuation handle 150, for insertion of respective attachment pins 164a, 164b of the drive wires 210 into respective clamping mechanisms (FIG. 6B). A guide rod may be moved provided in a center of the attachment pins to facilitate alignment of the catheter hub 170 with the actuation handle 150.

FIG. 7B illustrates the catheter hub 170 joined with the actuation handle 150, and an initial rotation being performed of the attachment collar 160. In FIG. 7B, the attachment collar 160 is rotated in the securing direction from the hub release zone 560 into the wire disengagement zone 540.

In FIG. 7C, the attachment collar 160 is further rotated in the securing direction from the wire disengagement zone 540 into the inaction zone 510. FIG. 7D illustrates the catheter hub 170 fully joined with the actuation handle 150 after the attachment collar 160 is fully rotated in the securing direction into the inaction zone 510, rendering the actuation handle 150 ready for initial a medical procedure.

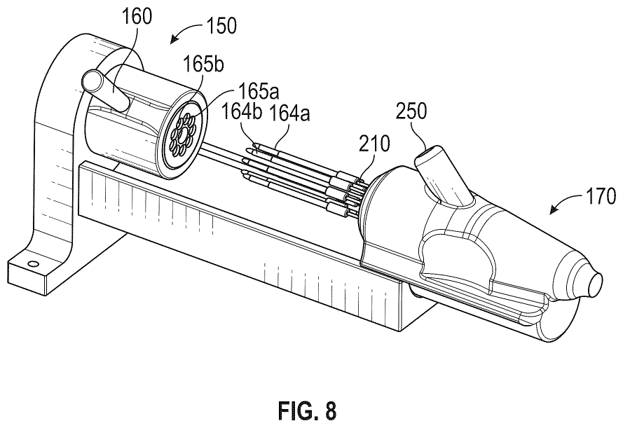

FIG. 8 illustrates a shelf mechanism to assist aligning the catheter hub with the attachment collar. As illustrated in FIG. 8, the shelf mechanism is axially provided for positioning and aligning the catheter hub 170 with the attachment collar 160.

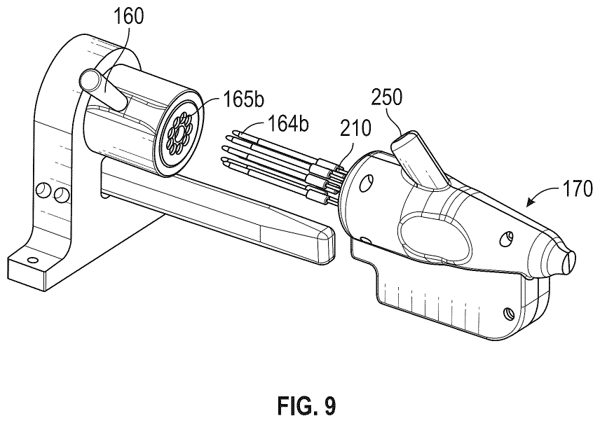

FIG. 9 illustrates a rail mechanism for aligning the catheter hub with the attachment collar. As illustrated in FIG. 9, the rail mechanism is axially provided for positioning and aligning the catheter hub 170 with the attachment collar 160.

The shelf mechanisms of FIGS. 8 and 9 facilitate alignment and expedited insertion of the catheter hub 170 onto the actuation handle 150, and avoid non-alignment of the attachment pins 164a, 164b with respective clamping mechanisms 165a, 165b.

FIGS. 10A to 10D illustrate a process for detaching the catheter hub 170 from the actuation handle 150, according to an embodiment.

In FIG. 10A, the catheter hub 170 is attached to the actuation handle 150, and the attachment collar 160 begins rotation in the release direction from the inaction zone 510 to the detent positioned at the second end 516 of the inaction zone 510. The attachment collar 160 is freely rotatable within the inaction zone 510. That is, movement of the collar in the inaction zone 510 does not trigger any state change, e.g., disconnect of attachment pins 164a, 164b from respective clamping mechanisms 165a, 165b. Thus, a user may freely rotate in the release direction or the securing direction, while in the inaction zone 510, i.e., between the first end 512 and the second end 516 of the inaction zone 510, without consequence.

In FIG. 10B, after overcoming the detent at the second end 516 of the inaction zone 510 the attachment collar 160 is rotated in the release direction into the wire disengagement zone 540. While in the wire disengagement zone 540, distal ends of the plurality of drive wires 210 are in a disengaged mode. The wire disengagement zone 540 is configured to simultaneously disengage at least three drive wires of the medical apparatus actuator 310. Thus, in the disengaged mode, the catheter is in a flexible emergency mode. In the emergency mode, the attachment collar 160 is easily turn, i.e., easy to rotate, to disengage the drive wires 210 and enhance safety during surgery, including robotic surgery.

FIG. 10C illustrates a user depressing the release mechanism 550, to overcome the second hard stop that is provided at the second end 546 of the wire disengagement zone 540.

FIG. 10D illustrates rotation into the release direction past the second end 546 of the wire disengagement zone 540, into the hub release zone 560. Rotation of the attachment collar 160 into the hub release zone 560 initiates decoupling of the drive wires 210 from drive mechanisms that are provided in the actuation handle 150. Rotating the attachment collar 160 through the hub release zone 560 to a third (final) hard stop 564 releases the attachment pins 164a, 164b from the respective clamping mechanisms 165a, 165b that are provided in the actuation handle 150. Thus, the user may axially slide the catheter hub 170 away from the actuation handle 150, to separate the actuation handle 150 from the catheter hub 170.

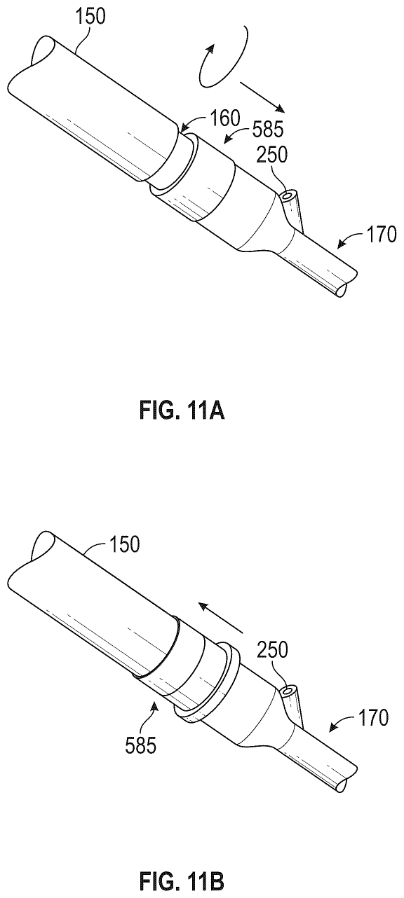

FIGS. 11A and 11B illustrate a sleeve guard 585, according to an embodiment. The sleeve guard 585 may axially slide into a closed position to cover a portion or all of the attachment collar 160. As illustrated in FIG. 11A, when in an open orientation, the sleeve guard 585 allows access to the attachment collar 160, thereby allowing rotation of the attachment collar 160. As illustrated in FIG. 11B, when in a closed orientation, the sleeve guard 585 precludes access to the attachment collar 160, thereby preventing rotation of the attachment collar 160.

FIG. 12 illustrates operation of a release mechanism, according to an embodiment. Squeezing the release mechanism 550 allows the drive wires 210 of a steerable catheter 100 to release from the catheter hub 170. As illustrated in FIG. 12, the release mechanism 550 may be a dual-sided button requiring compression from each side for operation thereof. Thus, linear, rather than rotational, control is provided for release of the drive wires 210 from the catheter hub 170.

Operation of, i.e., depressing, the release mechanism 550 may provide linear motion for controlled travel from/to the inaction zone 510 into/from the wire disengagement zone 540 and/or from/to wire disengagement zone into/from the hub release zone 560. For linear control, travel of approximately one quarter to a half inch may facilitate travel through the inaction zone 510, travel of approximately a half inch to two inches may facilitate travel through the wire disengagement zone 540, and travel of approximately one inch may facilitate travel through the hub release zone 560, with travel through the hub release zone 560 may only be accessible after pressing an interlock button that allows final separation between the actuation handle 150 and the catheter hub 170.

A locked state sensor 305 (FIGS. 7B-7C) detects movement between a locked state and an unlocked state of the medical apparatus actuator 310 with the catheter hub 170. One or more rotation sensors may also be provided, for example position sensors 304a, 304b, 304c, 304d. As shown in FIG. 5, a first position sensor 304a may be provided at the first end of the inaction zone 512, a second position sensor 304b may be provided at the first end of the wire disengagement zone 542, a third position sensor 304c may be provided at the second end of the wire disengagement zone 546 and a fourth position sensor 304d may be provided at the third hard stop 564, thereby providing output of rotational status of the attachment collar 160. Output of one or more of the locked state sensor 305 and/or the position sensors 304a, 304b, 304c, 304d may be displayed on the display screen 420. Detection of rotation into the hub release zone 560 may pause operation of the robotic catheter system 1000.

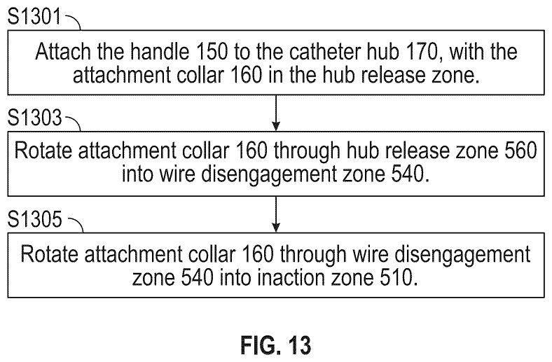

FIG. 13 illustrates a method for securing a catheter to an actuator of a medical apparatus, according to an embodiment. In FIG. 13, the catheter includes a catheter hub and the actuator includes a handle and an attachment collar operable through an inaction zone, a wire disengagement zone, and a hub release zone.

In step S1301 of FIG. 13, the handle 150 is attached to the catheter hub 170, with the attachment collar 160 in the hub release zone. In step S1303, the attachment collar 160 is rotated through the hub release zone 560 into the wire disengagement zone 540. In step S1305, the attachment collar 160 is rotated through the wire disengagement zone 540 into the inaction zone 510. Rotating through the wire disengagement zone 540 secures attachment pins 164a, 164b extending from the catheter hub 170 to respective clamping mechanisms 165a, 165b of the actuator.

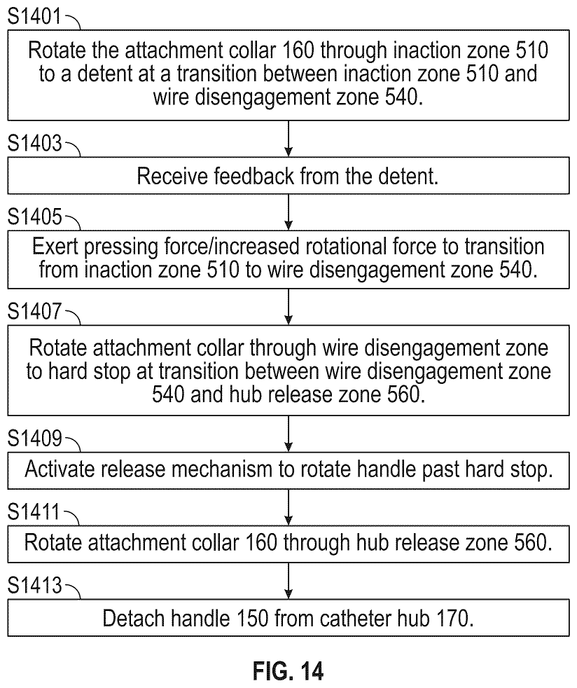

FIG. 14 illustrates a method for releasing a catheter from an actuator of a medical apparatus, according to an embodiment. In FIG. 14, the catheter includes a catheter hub and the actuator includes a handle and an attachment collar operable through an inaction zone, a wire disengagement zone, and a hub release zone.

In Step S1401 of FIG. 14, a rotational force is exerted on the attachment collar 160 to rotate the attachment collar 160 through the inaction zone 510 to a detent provided at a transition between the inaction zone 510 and the wire disengagement zone 540. In Step S1403, feedback is received from the detent. In Step S1405, in response to the feedback, one of a pressing force and an increased rotational force is exerted to transition from the inaction zone 510 to the wire disengagement zone 540.

In Step S1407, the attachment collar 160 is rotated through the wire disengagement zone 540 to a hard stop provided at a transition between the wire disengagement zone 540 and the hub release zone 560. In Step S1409, rotation continues past the hard stop by activating a release mechanism. In Step S1411, the attachment collar 160 is rotated through the hub release zone 560 to release attachment pins 164a, 164b extending from the catheter hub 170 from respective clamping mechanisms 165a, 165b of the actuator. In Step S1413 the handle 150 is detached from the catheter hub 170.

The attachment collar and method of use provide advantages that include one or more of: providing a zone of rotation that precludes initiation of any action while absorbing accidental rotation; control of wire disengagement and attachment of the catheter hub to the actuation handle, while providing various feedback indicating initiation/completion of predetermined actions; providing a final safety control permitted removal of the catheter, providing additional disengagement safety to avoid procedural delay; and receiving input from the rotation detection sensor for pause of the robotic system to avoid potential restart of an ongoing procedure.

Reference numbers:

| | |
|---|---|
| 10 | user |
| 50 | connector assembly |
| 60 | EM field generator |
| 80 | patient |
| 81 | bodily lumen |
| 90 | robotic platform |
| 91 | translation linear stage |
| 92 | robotic arm |
| 100 | steerable catheter |
| 104 | conduits |
| 105 | tool channel |
| 108 | wire-guiding members |
| 109 | anchor members |
| 110 | robotic catheter |
| 120 | catheter tip |
| 130 | distal section |
| 140 | proximal section |
| 150 | actuation handle |
| 160 | attachment collar |
| 161 | external user cylinder |
| 162 | internal cylinder |
| 164a, b | attachment pins |
| 165a, b | clamping mechanisms |
| 170 | catheter hub |
| 180 | imaging device |
| 181 | target |
| 190 | EM sensor(s) |
| 205 | handheld controller |
| 210 | drive wires |
| 250 | access ports |
| 300 | actuator system |
| 304a . . . d | position sensors |
| 305 | locked state sensor |
| 310 | actuator |
| 320 | micro-controller |
| 400 | computer |
| 410 | CPU |
| 411 | memory |
| 412 | system interface |
| 413 | user interface |
| 420 | display screen |
| 421 | graphical representation |
| 422 | endoscope (live view) image |
| 423 | intra-operative guiding image |
| 424 | pre-operative image |
| 425 | network connection |
| 510 | inaction zone |
| 512 | first end of inaction zone |
| 516 | a second end of inaction zone |
| 540 | wire disengagement zone |
| 542 | first end of wire disengagement zone |
| 546 | second end of the wire disengagement zone |
| 550 | release mechanism |
| 560 | hub release zone |
| 562 | first end of the hub release zone |
| 564 | final hard stop |
| 1000 | robotic catheter system |

In referring to the above description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. An attachment collar configured to detachably connect a medical apparatus actuator to a catheter hub, the attachment collar comprising:
an inaction zone;
a wire disengagement zone; and
a hub release zone.

2. The attachment collar of claim 1, wherein the inaction zone comprises a first end and a second end opposite the first end.

3. The attachment collar of claim 2, wherein the attachment collar is configured to rotate between the first end and the second end of the inaction zone without triggering a state change.

4. The attachment collar of claim 2, wherein the first end of the inaction zone comprises a hard stop.

5. The attachment collar of claim 2, further comprising a detent provided at the second end of the inaction zone, wherein, upon rotation of the attachment collar to the second end of the inaction zone, the detent is configured to output feedback to a user of the attachment collar.

6. The attachment collar of claim 5, wherein the feedback is at least one of a sound, a visual indicator, and an increase in rotational force to continue to rotate the attachment collar.

7. The attachment collar of claim 2, wherein:
a first end of the wire disengagement zone is adjacent to the second end of the inaction zone, and
a first end of the hub release zone is adjacent to a second end of the wire disengagement zone.

8. The attachment collar of claim 7, wherein the second end of the wire disengagement zone comprises a hard stop.

9. The attachment collar of claim 8, wherein the hard stop includes a release mechanism.

10. The attachment collar of claim 9, wherein activation of the release mechanism permits rotation of the attachment collar past the hard stop.

11. The attachment collar of claim 9, wherein the release mechanism comprises a push button.

12. The attachment collar of claim 7, wherein, rotation of the attachment collar from the second end of the wire disengagement zone through the hub release zone releases attachment pins of a catheter operably connected to the catheter hub.

13. The attachment collar of claim 7, wherein rotation of the attachment collar from the second end of the wire disengagement zone through the hub release zone releases at least one drive wire of the medical apparatus actuation.

14. The attachment collar of claim 7, wherein the medical apparatus actuator is configured to be removable from the catheter hub upon rotation of the attachment collar past the second end of the wire disengagement zone.

15. The attachment collar of claim 1, further comprising a release mechanism, wherein operation of the release mechanism allows for release of at least one drive wire of a catheter operably connected to the catheter hub.

16. The attachment collar of claim 1, further comprising a sensor configured to detect one of a locked state and an unlocked state of the medical apparatus actuator with the catheter hub.

17. The attachment collar of claim 1, further comprising a sleeve guard configured to slide axially between an open orientation and a closed orientation, wherein:
when in the open orientation, the sleeve guard allows access to the attachment collar, and
when in the closed orientation, the sleeve guard precludes access to the attachment collar.

18. The attachment collar of claim 1, wherein the attachment collar is movably affixed to an actuation handle of a robotic catheter system.

19. A method for securing a catheter to an actuator of a medical apparatus, with the catheter including a catheter hub, and with the actuator including a handle and an attachment collar operable through an inaction zone, a wire disengagement zone, and a hub release zone, the method comprising:
attaching, with the attachment collar in the hub release zone, the handle to the catheter hub;
rotating the attachment collar through the hub release zone into the wire disengagement zone; and
rotating the attachment collar through the wire disengagement zone into the inaction zone,
wherein the rotating through the wire disengagement zone secures attachment pins extending from the catheter hub to respective clamping mechanisms of the actuator.

20. A method for releasing a catheter from an actuator of a medical apparatus, the catheter including a catheter hub; the actuator including a handle and an attachment collar operable through an inaction zone, a wire disengagement zone, and a hub release zone;
the method comprising:
exerting a rotational force on the attachment collar to rotate the attachment collar through the inaction zone to a detent provided at a transition between the inaction zone and the wire disengagement zone;
receiving feedback from the detent;
in response to the feedback, exerting one of a pressing force and an increased rotational force to transition from the inaction zone to the wire disengagement zone;
rotating the attachment collar through the wire disengagement zone to a hard stop provided at a transition between the wire disengagement zone and the hub release zone;
rotating past the hard stop by activating a release mechanism;
rotating the attachment collar through the hub release zone, wherein the rotating of the attachment collar through the hub release zone releases attachment pins extending from the catheter hub from respective clamping mechanisms of the actuator; and
detaching the handle from the catheter hub.
